(19) United States
(12) Patent Application Publication
(10) Pub. No.: US 2006/0024195 A1
(43) Pub. Date: Feb. 2, 2006

(54) Non-thermal disinfestation of biological pests with pulsed radio frequency power systems

(75) Inventors: Manuel C. Lagunas-Solar, Davis, CA (US); Timothy K. Essert, Sacramento, CA (US)

Correspondence Address: JOHN P. O'BANION;O'BANION & RITCHEY LLP, 400 CAPITOL MALL SUITE 1550, SACRAMENTO, CA, 95814, US

(73) Assignee: THE REGENTS OF THE UNIVERSITY OF CALIFORNIA

(21) Appl. No.: 10/900,990

(22) Filed: Jul. 27, 2004

(51) Int. Cl. A61L 2/08; A61L 2/12

(52) U.S. Cl. 422/22; 422/21

(57) ABSTRACT

An apparatus and method for disinfestation of temperature sensitive and other commodities using short duration, high peak power radio frequency pulses and intense electric fields to preferentially induce drift of conduction charges, spot heating and other mortal damage to infesting insects and mites without raising the temperature of the host commodity. A low frequency mode is provided with a frequency range of approximately 10 Hz to 1 MHz and is particularly suited for large batch processing of pallets or field containers. A high frequency mode is also provided that is suited for continuous or small batch processing and uses a preferred frequency range of approximately 1 MHz to 150 MHz. Both modes preferably have an electric field strength of greater than approximately 5 kV/cm and pulse repetition rates of between one pulse and approximately 10 million pulses per second. The method is non-contact, residue free and effective with all biological stages of an infesting insect, mite or other biological pest including egg, pupa, larvae, juvenile and adult forms. The apparatus and methods of the invention are an effective alternative to methyl bromide fumigation that does not leave any toxic residues or damage the cosmetic appearance or flavor of the commodity.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to devices and schemes for disinfestation of insects, arachnids and other biological pests, and more particularly to a non-thermal disinfestation apparatus and method using pulsed radio frequency power systems.

2. Description of Related Art

The longevity of perishable commodities in the marketplace is often diminished by damage caused by infestations of insects, mites and other biological pests. The presence of egg, larval and adult forms of insect pests creates the possibility of cross-infestation of commodities and progressively increasing losses during storage or transportation of commodities to market. Transportation of insects and other pests on host commodities across state and country boundaries artificially increases the natural range of such pests and may introduce pests into areas that allow the pests to have a selective advantage over indigenous insects or to become a threat to local crops.

Each year, considerable quantities of pesticides are applied to commodities by producers at various stages of agricultural production, from pre-planting to post harvest, in order to eradicate unwanted insects and other animal, microbial and fungal pests. Established quarantine barriers regulate the transportation of agricultural commodities worldwide in order to reduce the potential for propagating and transporting non-indigenous pests. Many commodities cannot be legally imported or exported without pesticide treatments to eliminate quarantine pests and to certify that the commodities are free from pests.

Methyl bromide, for example, is widely used in the industry as a gaseous fumigant that can disinfest a variety of fresh foods, agricultural soils and structural facilities. However, it is anticipated that methyl bromide will be banned because of the capability of methyl bromide to scavenge ozone in the atmosphere. Agriculture in the United States used an average of about 60 million pounds of methyl bromide per year before the mandatory reductions began in 1999. The use of pesticides in general and insecticides in particular are of global concern due to detrimental effects on animals, air, water and soil as well as the impact they have on public health and agricultural workers.

The use of methyl bromide or other chemicals in the fresh fruit industry is often unsatisfactory because chemical use may create cosmetic blemishes or reduce the effective shelf life of the fruit. In addition, applications of methyl bromide at concentrations sufficient to control pests on stored and exported commodities may produce bromide residue levels that are relatively high.

Likewise, other pesticides known in the art have shown erratic performance at low concentrations and have produced crop damage and unacceptable residue levels in some cases. Other pesticides commonly used in pre-harvest and post-harvest applications include phosphine, chloropicrin, 1,3-dichloropropene, Telone/Vapam, sulfaryl fluoride and hydrogen cyanide.

Another approach to the eradication of insect infestation in food commodities in the art is the use of thermal energy. However, thermal energy, such as the use of hot water, is unsatisfactory because it can cause rapid deterioration of the color, texture and flavor of a food commodity and typically uses a large amount of energy. Spot heating of a commodity may also produce discolorations and change the characteristics and shelf life of a commodity. Thermal energy is normally used in the fresh produce industry only when no other alternatives are viable or available.

High intensity light has been used with some success for surface de-contamination in prepackaged foods. Absorption of ultraviolet light by a pest, for example, causes lethal damage to the organism by conversion of the ultraviolet light into thermal energy. However, high intensity light has been less successful with insect and mite pests on fresh food commodities because of the need to expose all surfaces to the light and the commodities cannot be treated efficiently in bulk.

A further approach to disinfestation is through the use of radioactive materials or electron beam accelerators to provide gamma ray, X-ray or other electron radiolytic effects in pests. However, although commercial systems are available, the use of radiation based systems are opposed by portions of the general public who do not want to eat irradiated foods.

In the United States, as well as in many other countries, changes in public attitudes towards the use of chemicals and radiation to control pests have resulted from increased concern for food safety and the preservation of environmental quality. Popular awareness and attitudes concerning these problems are reflected in the increasing number of regulatory actions by governments targeting agricultural pesticides. As the regulations have increased, the availability of agricultural pesticides has decreased. This has imposed new technological demands on agriculture and may create new barriers to the international trade of foods and agricultural commodities because of the existence of quarantine regulations between trading partners.

Therefore, there is a need for an apparatus and methods for efficiently and effectively destroying insects in food commodities without leaving chemical residues and altering the characteristics of the commodity that is treated. The present invention satisfies that need, as well as others, and overcomes the deficiencies in prior insect control technologies.

### BRIEF SUMMARY OF THE INVENTION

The present invention generally pertains to an apparatus and method of exposing insect pests and other undesirable organisms found in food or other commodities to pulsed radio frequency energy wherein the undesirable organisms are selectively eradicated without substantially raising the temperature or causing damage to the commodity.

By way of example, and not of limitation, the method of the present invention generally comprises the steps of exposing insects and other organisms to a plurality of high intensity, high frequency electromagnetic energy pulses in the radio frequency spectrum. Pulsed radio frequency power has been shown to disinfest fresh fruits with a non-thermal, fast, effective, non-contact and energy-efficient process. Immediate and delayed mortality, or biological injuries leading to sterilization reaching greater than 99%, has been demonstrated in fresh fruits and with all the biological stages (i.e., adults, larvae, pupas, or eggs) of a variety of insects and mites.

Radio frequency (RF) radiation refers to electromagnetic radiation in the frequency range from approximately 1 hertz to 300 gigahertz. Preferred frequencies are in the range of approximately 10 Hz to approximately 150 MHz (preferably 10 to 100 Hz for batch processing; 5 to 40 MHz for on-line processing), with electrical fields greater than or equal to 5 kV/cm, and repetition rates up to approximately ten kilohertz, are used to disinfest commodities using, for example, a well-insulated conventional parallel-plate metallic cavity.

The RF disinfestation method according to the invention can operate within two distinct sets of operating parameters; the first using frequencies ranging from approximately 10 Hz to 1 MHz (arbitrarily defined as low frequency) and the second using low to high frequencies ranging from approximately 1 MHz to 150 MHz (arbitrarily defined as high frequency).

Each set of conditions can provide disinfestation effects based upon drifting, but in different time sequences and product geometries. The electromagnetic radio frequency energy generates lethal and some sub-lethal effects due to electric polarization induced preferentially and selectively on insects and mites. The disinfestation effect is based on applying an oscillating high-intensity electrical field that causes drifting or movement in conduction charges. The drifting motion transfers energy to critical cellular and sub-cellular structures and/or causes extremely rapid resistance heating within insects and mites. Under these conditions, non-thermal direct effects are optimized while other mechanisms leading to dielectric heating (i.e. production of heat), are avoided or largely minimized.

The Low Radio frequency Disinfestation (Low-RFD) process is suitable for processing commodities in large volumes in a batch mode (including pallets and other large volume containers) in processes lasting from minutes to hours. The High Radio frequency Disinfestation (High-RFD) process is suited for on-line processing with processing times ranging from a few seconds to tens of seconds to minutes in thinner containers. The processing times are primarily dependent upon the frequency and pulse repetition rate.

Periodic (non continuous or alternating) pulses of RF energy are preferably delivered within a parallel-plate cavity using frequencies, pulse shapes, duration and repetition rates that deliver RF power in a manner that causes no detectable thermal energy production in the host commodity. The RF power may be pulsed with extremely short duration times (nano to microseconds) and may be applied with repetition rates ranging from 1 Hz to approximately 10 kHz. Pulses are preferably formed with rapid rise and fall times while pulse duration is maintained as short as possible (i.e. less than milliseconds). Pulses with sinusoidal shapes are preferred but pulses with other forms are also adequate. Pulses with a square-wave form are preferred over other types of pulses including, but not excluding, exponential, bipolar, and oscillatory pulses.

It has also been shown that radio frequency energy can create ozone or oxygen radicals near the surface of treated commodities that can have concurrent and synergistic effect on the eradication of insects and mites. Ozone provides a secondary toxic effect on all forms of insects and mites along with the direct detrimental effects of high intensity radio frequency pulses.

The method and apparatus may also provide for commodity treatments that have multiple conditions created in succession. For example, a first stage provides a treatment with an RF frequency, field strength, pulse repetition rate and time parameters that are optimized to the creation of ozone. Subsequent stages may provide treatment conditions that are optimized for eradicating a particular pest in the egg, larval or adult forms.

Accordingly, a non-contact, controlled thermal, chemical free apparatus and method are provided that can disinfest a commodity from insects, mites or other animals that can provide quarantine level eradication without destroying the shelf life, physical attributes or quality of the host commodity.

In one beneficial embodiment, a method for disinfestation of a commodity according to the present invention comprises exposing an infested commodity to a non-thermal, pulsed, high intensity electric field radio frequency for less than approximately 24 hours. In one mode, the radio frequency comprises a low frequency ranging from approximately 10 Hz to approximately 1 MHz. In another mode, the radio frequency comprises a high frequency ranging from greater than approximately 1 MHz to approximately 150 MHz. According to one aspect of the invention, the pulsed radio frequency has a repetition rate ranging from approximately 1 Hz to approximately 10 KHz. According to another aspect of the invention, the pulses have short duration times of less than one second. In accordance with a still further aspect of the invention, the pulses have rapid rise times and short decay times whereby RF peak power can be maximized. In one mode, the duty cycle, electric field strength and frequency are optimized to the sensitivity of a pest to exposure to radio frequency energy. In another mode, the duty cycle, electric field strength and frequency are optimized to the sensitivity of a pest and a host commodity to exposure to radio frequency energy.

In another beneficial embodiment, a method for disinfesting biological pests from a commodity according to the invention comprises exposing an infested commodity to a first high intensity radio frequency for a first duration, and exposing the commodity to a second high intensity radio frequency for a second duration, wherein the temperature of said commodity does not substantially change during exposure. According to one aspect of the invention, the radio frequencies are pulsed. According to another aspect of the invention, the pulsed radio frequencies have a repetition rate ranging from approximately 1 Hz to approximately 10 KHz. According to another aspect of the invention, the pulses have short duration times of less than approximately one second. In accordance with a still further aspect of the invention, the pulses have rapid rise times and short decay times whereby RF peak power can be maximized.

In one mode, the first and second radio frequencies comprise low frequencies ranging from approximately 10 Hz to approximately 1 MHz. In another mode, the first and second radio frequencies comprise high frequencies ranging from greater than approximately 1 MHz to approximately 150 MHz. According to one aspect of the invention, the first radio frequency comprises a low frequency ranging from approximately 10 Hz to approximately 1 MHz, and the second radio frequency comprises a high frequency ranging from greater than approximately 1 MHz to approximately 150 MHz. According to another aspect of the invention, the first radio frequency comprises a high frequency ranging from approximately 1 MHz to approximately 150 MHz, and the second radio frequency comprises a low frequency ranging from approximately 10 Hz to less than approximately 1 MHz. In one mode, the duty cycle, electric field strength and frequency of said first radio frequency exposure are optimized to the sensitivity of an adult form of a pest to exposure to radio frequency energy. In another mode, the duty cycle, electric field strength and frequency of said first radio frequency exposure are optimized to sensitivity of a larval form of a pest to exposure to radio frequency energy. In another mode, the duty cycle, electric field strength and frequency of said first radio frequency exposure are optimized to sensitivity of an egg form of a pest to exposure to radio frequency energy. In another mode, the duty cycle, electric field strength and frequency of said first and second frequency exposures are optimized to sensitivity of a pest and a host commodity to exposure to radio frequency energy. In another mode, the first radio frequency is optimized to disinfest egg forms of a pest and said second radio frequency is optimized to disinfest adult forms of a pest. In another mode, the first radio frequency is configured to produce ozone, and the second radio frequency is optimized to disinfest adult forms of a pest.

In a further beneficial embodiment, a method for residue free disinfestation of an article according to the invention comprises treating an article with pulsed high intensity radio frequency electromagnetic radiation at a plurality of different frequencies, and controlling the temperature of said article over a course of treatment. In one mode, the temperature of said article is controlled by modulating the radio frequency, pulse rate or electric field intensity during treatment. In another mode, the article is treated with high intensity radio frequency electromagnetic radiation that has an electric field intensity of greater than approximately 5 kilovolts per centimeter. According to one aspect of the invention, the radio frequency comprises a radio frequency and an electric field strength configured to produce ozone and oxygen radicals from ambient air. According to another aspect of the invention, the exposure to electromagnetic radiation at a plurality of radio frequencies comprises exposure to a first radio frequency for a first duration, exposure to a second radio frequency for a second duration, and exposure to a third radio frequency for a third duration. In one mode, the first, second and third radio frequencies comprise a low frequency ranging from approximately 10 Hz to approximately 1 MHz. In accordance with another aspect of the invention, the first, second and third radio frequencies comprise a high frequency, said high frequency ranging from approximately 1 MHz to approximately 150 MHz. In another mode, the first radio frequency comprises a low frequency, said low frequency ranging from approximately 10 Hz to approximately 1 MHz, and the second radio frequency comprises a high frequency, said high frequency ranging from approximately 1 MHz to approximately 150 MHz.

An aspect of the invention is to provide a chemical free disinfestation method that is an alternative to the use of chemical pesticides including methyl bromide.

Another aspect of the invention is to provide a non-thermal or controlled thermal, non-contact disinfestation method based upon pulsed radio frequency power.

Another object of the invention is to provide a method and apparatus that will disinfest all biological stages of insects and mites including adults, larva and eggs.

A still further aspect of the invention is to provide an apparatus that uses high-peak pulsed RF power with oscillating electric fields that are greater than or equal to approximately 5 kV/cm.

Another aspect of the invention is to provide a method and apparatus that uses RF pulses with rapid rise times and short decay times to maximize RF peak power.

Another aspect of the invention is to provide an apparatus and method that permits the selection of an electric field intensity that maximizes drifting mechanisms in conductive insects and mites while minimizing electric polarization effects in dielectric host materials.

Still another aspect of the invention is to provide an apparatus and method that provides RF pulses that range from approximately ten nanoseconds to approximately one tenth of a second in duration.

Yet another aspect of the invention is to provide a method and apparatus that combines uniquely higher frequencies and electric-field intensities, with minimal generation of unwanted harmonics, while achieving high voltages in small volumes and power efficiencies.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWINGS

The invention will be more fully understood by reference to the following drawings which are for illustrative purposes only:

FIG. 1 is a functional block diagram of one embodiment of a method for disinfestation using radio frequency exposures according to the present invention.

FIG. 2 is a schematic diagram of a batch-processing embodiment adapted for a single pallet according to the present invention.

FIG. 3 is a schematic diagram of a batch-processing embodiment adapted for multiple pallets according to the present invention.

FIG. 4 is a schematic diagram of a High Electric Field Pulsed RF processing apparatus embodiment adapted for batch or on-line processing according to the present invention.

FIG. 5 is a perspective view of an online processing embodiment of the present invention.

### DETAILED DESCRIPTION OF THE INVENTION

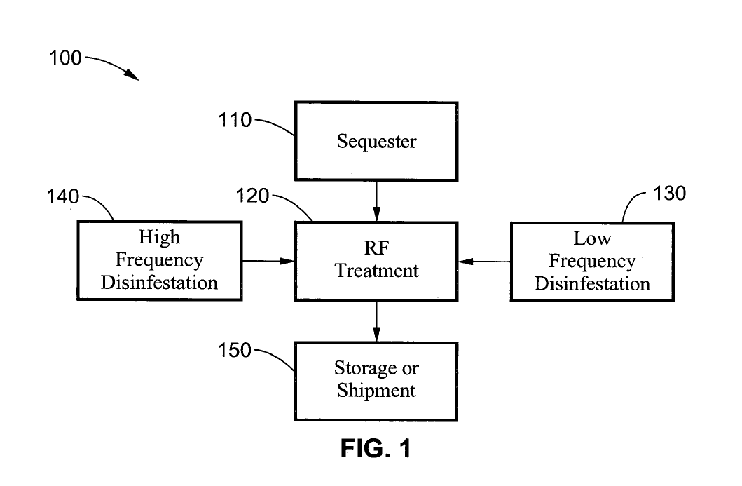

Referring more specifically to the drawings, for illustrative purposes the present invention is embodied in the apparatus and methods generally shown in FIG. 1 through FIG. 5. It will be appreciated that the apparatus may vary as to configuration and as to details of the parts, and that the method may vary as to the specific steps and sequence, without departing from the basic concepts disclosed herein.

The apparatus and methods of the present invention may be particularly beneficial to the disinfestation of thermally sensitive commodities such as fresh produce including fruits and vegetables, ornamental and cut flowers and nursery stocks. Perishable food commodities are often very difficult to disinfest with existing approaches in the art because of changes created in the physical or chemical properties of the commodity or by the deposition of toxic residues by their use. The methods of the present invention may also be adapted for disinfestation of commodities that are not thermally sensitive such as dried foods, nuts, grains, seeds, cereals, and animal feeds as well as eggs, wood products, and nursery or containerized soils and other non food commodities. The apparatus and methods of the invention are an effective alternative to methyl bromide fumigation that does not leave any toxic residues or damage the cosmetic appearance or flavor of the commodity.

Turning now to FIG. 1, a flow chart of one embodiment of the disinfestation method 100 using radio frequency exposures is generally shown. At block 110, the commodity is preferably sequestered in a container or enclosure to isolate the commodity for disinfestation. Isolation restricts the movement of mobile pests so that the pests will be exposed to the lethal effects of the process and limits the occurrence of re-infestation or contamination from one lot to another within a warehouse, packing plant or long term storage facility. While the optional placement of the commodity in a container at block 110 is preferred, it will be understood that the RF treatment can be administered to commodities that are on pallets, tubs, vats and other open receptacles. Sequestration may also be achieved by placing a top on a storage container from the field. The container is preferably made of material that does not substantially resist the passage of radio waves through the container and does not conduct electricity. Accordingly, a wide variety of containers may be used.

The commodities receive RF treatment at block 120 and then are stored or shipped at block 150. The RF treatment provided at block 120 can be applied using either a low frequency disinfestation method 130 or a high frequency disinfestation method 140. It will be seen that the RF treatments at block 120 can be single treatments using the high or low frequency disinfestation method or can be a succession of treatments using different parameters.

Generally, the apparatus and methods of the invention use radio frequency power to disinfest normal and thermally sensitive commodities with a non-thermal, energy-efficient and non-contact process. With fresh fruits, for example, the process eliminates the potential for inducing detrimental thermal effects that reduce the quality and condition of the fruit and limit the shelf life and market value of the commodity. It will be seen that disinfestation of a commodity from insects and mites, reaching greater than approximately 99%, can be achieved without any detectable levels of thermal energy in the host commodity and without the use of chemicals. The method is effective on all the biological stages (i.e., adults, larvae, pupas, or eggs) of a variety of insects, mites and other pests. The method is based upon the differential sensitivity of insects and mites to exposure to static or pulsed radio frequency power as compared to the host commodity. The differential sensitivity of pests is due to the higher biochemical and metabolic complexity of the more differentiated organisms such as insects and mites verses the less sensitive, biochemically simpler host commodity.

One way to meet the objective of delivering RF power that is lethal to insects and mites without inducing thermal energy in the host commodity is by using a pulsed RF source. It will be seen that the duration, repetition rate and other pulse characteristics can be manipulated to maximize the lethal effects on insects while minimizing the exposure of the commodity to thermal energy.

When using a pulsed RF source, the duty cycle (D_{C}) is a relevant quantity. The duty cycle is defined as the ratio of the pulse duration (P_{D}) to the pulse repetition time (P_{RT}) of a periodic sequence of pulses. The pulse repetition time (P_{RT}) is defined as the reciprocal of the pulse repetition frequency (P_{RF}) (also known as pulse repetition rate), which is given by the number of pulses per unit time. These definitions provide the following expression:
D_{C}=P_{D}/P_{RT}=P_{D}×P_{RF}

Because high peak powers are delivered with a pulsed RF source, it is important that the duty cycle (D_{C}) that is used is appropriate to deliver enough RF energy to overcome the threshold energy required for inducing lethal and sub lethal damage to insects and mites, while operating below the thresholds required for the host commodity to absorb RF energy and convert it to thermal energies.

Since the duty cycle (D_{C}) can be controlled with the pulse repetition frequency, a pulsed RF system can be adapted to operate with appropriate frequency, pulse shape, pulse duration, pulse repetition rate, duty cycle, and pulse uniformity, in order to deliver over a fairly short treatment time (few seconds to minutes) and a sufficient number of pulses with sufficient peak power (preferably greater than 100 kW) and intense electric fields (preferably greater than 5 kV/cm) to disinfest the commodity. Each of these parameters can be adjusted to optimize the lethal effects on a wide range of pests on a wide variety of commodities. For example, simple experimentation and experience with the adult, larval and egg stages of each identified species of pest will allow the user to provide a duty cycle with pulses that will effectively eradicate the pest. Furthermore, short duration treatment times can be provided for commodities that are very sensitive to heat such as fruits, leafy vegetables and some historical artifacts and the like.

The electromagnetic radio frequency energy generates primarily lethal and some sub-lethal effects due to electric polarization that is induced preferentially and selectively on insects and mites. The disinfestation effect is based on applying an oscillating high-intensity electrical field that causes drifting in conduction charges. The drifting motion transfers energy to critical cellular and sub-cellular structures as well as causes extremely rapid resistance heating within insects and mites. Under these conditions, non-thermal direct effects on pests are optimized while other mechanisms leading to dielectric heating of the commodity are avoided.

Radio frequency fields interact and transfer energy to biological materials through the basic mechanisms of the polarization of bound charges, the orientation of permanent dipoles, and the drift of conduction charges (including electronic and ionic). The polarization of bound charges under an electric field consists of only a slight displacement of the charges within a biomaterial as restoring electrostatic forces tightly controls these charges. The net effect is to induce electric dipoles, which can be reoriented in the presence of an oscillating electric field. The new charge distribution (polarization charge) creates new fields and the friction between the induced dipoles and the surrounding media produces thermal energy. The effect is terminated when the electric field is cancelled.

The orientation of permanent dipoles in the presence of an electric field produces a slight reorientation as dipoles are randomly distributed due to strong forces derived from thermal excitation. Like the induced dipoles, the net alignment of the dipoles in the electrical field produces a new field while their friction causes heat.

The ability of electric fields to cause polarization and orientation effects is defined by a quantity known as permittivity, which is a measure of how easily the polarization and orientation changes occur in the presence of an electric field. Drift of conduction charges is defined by a quantity called conductivity, which is a measure of how much drift occurs in the presence of a given electric field. Complex conductivity also includes quantifying the drifting of dipoles.

The permittivity of biomaterials represents mostly ionic conductivity and absorption due to the relaxation processes, and includes friction associated with the alignment of electric dipoles and with vibration and rotational motion in molecules. Based upon the observed results and effects on insects and mites derived under these invention operational parameters, it appears that insects and mites have much greater conductivity and lower permittivity than fresh fruits and vegetables as no heat is generated on the latter while lethal effects are rapidly induced on insects and mites. By observation, the anatomical changes in the insect's morphology, such as abdomen expansion followed by contraction, wings bending and dry appearance, are all strong indicators of a thermally-induced injury mechanism leading to instant and delayed (<24 hours) mortality on insects and mites.

In addition to the drifting mechanism, electric conductivity effects that occur between the commodity's surface and the “conducting” insect or mites, causes a rapid and intense heating of insects. The resistance heating occurs according to Ohm's Law, a phenomena that has been observed experimentally as insects and mites appear charred under microscopic observations. These heating effects provide an effective and irreversible mechanism for mortality of insects and mites. The selective heating effect is enhanced by the instant (peak) RF power used in this method. However, because the total mass of the “heated” insects is a very small fraction of the total mass of the system (insect/mite with host commodity), the effect of radiation, conduction and/or convection heating of insects on the larger mass commodity is not significant and thus is undetectable.

Radio frequencies of suitable field strengths may also produce an important secondary effect that improves the efficiency of disinfestation of egg, larval and adult forms of infesting pests through the production of ozone or oxygen radicals. Ozone (O₃) is a powerful oxidizing agent that leaves no toxic residue and can be added or generated in situ to induce toxic effects to cellular metabolism in insects and mites. In situ generation also allows the use of the atomic oxygen precursor of molecular ozone as the initiator of the oxidizing effects of O₃. Atomic oxygen, an extremely rapid and reactive radical, is hundreds to thousands of times more reactive than molecular ozone.

Ozone may be formed near surfaces in air voids on commodities that are subjected to an oscillating electrical field that is generated by pulsed RF power. An electric field potential in air of between approximately 3 kV per centimeter to approximately 5 kV per centimeter is required to produce ozone at standard temperature and pressure. Ozone generated near the commodity surface provides a high probability for a direct effect on insects, mites, or on microbial contaminants present on the commodity surface.

Accordingly, it can be seen that the oscillating electric fields from a pulsed RF source not only causes the formation of drift and other direct lethal effects on insects and mites, it also can provide secondary lethal effects in the form of oxygen radicals and ozone. The method is believed to rapidly and irreversibly damage the nervous system and muscle activity in insects and mites. The combination of these effects can result in greater than 99% mortality instantly and reaching 100% mortality within a few hours due to delayed mortality due to injury.

The radio frequency disinfestation method shown in FIG. 1 can operate within two distinct sets of operating parameters or modes. The first set uses extremely low to low frequencies ranging from approximately 10 Hz to approximately 1 MHz that are defined as low frequencies. The second set uses low to high frequencies ranging from approximately 1 MHz to over 150 MHz and these frequencies are defined as high frequencies. Frequencies ranging from approximately 150 MHz to about approximately 250 MHz may be used with some thermally tolerant commodities. Radio frequency treatments are generated by electrical power being converted into very low frequency waves to very high frequency waves having a very broad range of wavelengths (i.e. 10 Hz=30,000 Km; 100 KHz=3,000 m; 150 MHz=2.0 m).

It will be appreciated that each set of conditions can provide physical effects based upon drifting in different time sequences and product geometries. For example, it is possible to formulate treatment schemes that have multiple stages with different treatment conditions at each stage. In one embodiment, the initial stage provides conditions to optimally produce ozone in situ. The second stage provides frequencies, field strengths and exposure time periods that are optimized to eradicate a particular adult pest. In another embodiment, a first stage provides treatment conditions that are optimized to eradicate eggs of an infesting pest while accounting for the sensitivity of the treated commodity. A second stage provides conditions that are optimized for larval forms with a third stage providing conditions that will eliminate adult forms.

Similarly, treatment schemes may be directed to disinfestation of multiple pests having different susceptibilities to RF exposure. These schemes would have stages providing treatment conditions that are optimized for each pest in various biological forms.

The selection of treatment conditions should take into account the sensitivity of the commodity to thermal and RF energy. Essentially all of the biological effects observed on a food commodity are related to the amount of energy absorbed by the commodity not the amount of exposure to an RF source. The selection of RF field properties such as frequency, intensity, pulse parameters, near verses far field and time rate of energy deposition preferably accounts for the dielectric values, dimensions, shapes, orientation and stability of a commodity within an electric field.

Thus, it will be seen that RF treatments according to the invention may be a single exposure to a single set of treatment conditions or sequential exposures to a variety of different treatment conditions to provide virtually complete disinfestation without the use of chemicals or damage to the commodity.

The first mode at block 130 of FIG. 1 is called Low Radio Frequency Disinfestation (Low-RFD) and is particularly suitable for processing large volumes in a batch processing system with pallets and other large volume containers and provides treatment times lasting from minutes to hours. The second mode 140 of FIG. 1 is called High Radio Frequency Disinfestation (High-RFD) and is particularly suitable for adaptation for on-line processing with processing times ranging from a few seconds to tens of seconds and minutes with the use of thin walled containers.

The time of exposure parameter is particularly dependent on the frequency and pulse repetition rate, while the choice of type of processing (batch or on-line) is primarily dependent on the ability of the apparatus to generate steady electric fields in appropriate capacitor gaps without corona discharge. Under the preferred operating conditions of either the High-RFD or Low RFD processes, the conductivity of insects allows for the use of a drift of conduction charges (electronic and/or ionic) that causes internal electric currents in the bodies of the pests. This is because insects and mites have certain characteristics of conductors and are subject to drifting effects, while host commodities are normally dielectrics. In addition, resistance-heating effects on contaminating insects and mites have been observed. The combination of the drifting (due to electric polarization) and heating effects provide sub-lethal as well as lethal injuries to insects and mites without generating thermal energy within the host commodities. Therefore, since the dielectric value of biomaterials increases with the exposed frequency, lower frequencies are preferred when processing thermally sensitive commodities.

Furthermore, it has been shown that the effectiveness of the pulsed RF disinfestation method is also enhanced by the conductivity of the host material, which increases from dried to fresh foods and can be altered by controlling the environmental conditions such as humidity and temperature of the commodity. Manipulations of the operational parameters will allow for shortened processing times and thereby allow materials to be processed on-line (bulk, boxes, etc.) or in larger pallet sized batches over comparatively longer periods of time from minutes to hours.

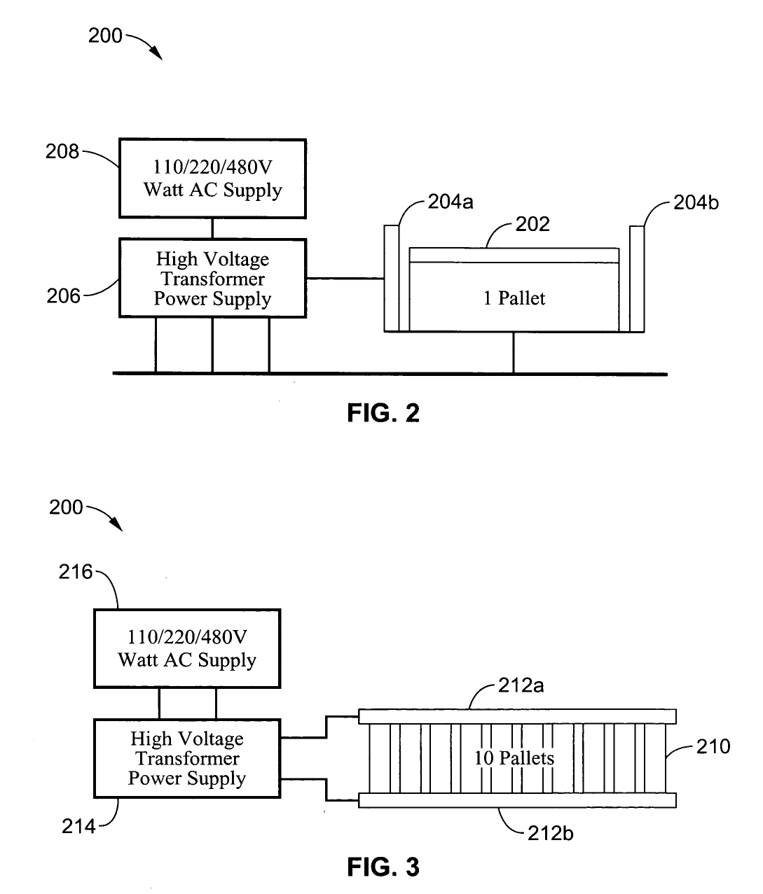

Referring also to FIG. 2 and FIG. 3, the Low-RFD method is particularly suited to batch processing type systems 200 using one or more pallets or large containers 202. The Low-RFD mode at block 130 of FIG. 1 preferably operates with frequencies of approximately 10 Hz to approximately 1 MHz with electric fields preferably greater than or equal to approximately 5 kV/cm.

The major advantage of the Low-RFD process is that with properly insulated capacitor plates 204a and 204b, large area capacitors are possible with large gaps greater than 1-meter allowing the disinfestation of large volumes of consumables or other materials on pallets or in field containers.

It can be seen that insulators are more effective at preventing corona discharges at lower frequencies due to capacitive effects rather than changes in dielectric strength. The electric field will deposit a charge on the surface of the insulated plate proportional to the strength of the electric field and the capacitance. The change in the charge occurs every cycle of the oscillation. This represents an alternating flow of electrons. The total number of electrons, in a unit time, flowing from the surface of one insulator, through the commodity, and to the surface of the opposite insulator is directly proportional to the frequency. The higher the frequency, the more electrons are flowing through the commodity, and the greater the risk of corona and electrical discharge.

Therefore, metallic capacitor plates 204a, 204b can be operated at very high voltages (i.e. megavolts) without corona discharges when covered with an appropriate layer of an insulator material of high dielectric strength such as Teflon, Glass, Polypropylene and the like. Therefore, commercially available power-line transformers 206 with conventional power supplies 208 can be used to generate adequate high voltages and electric fields greater than or equal to about 5 kV/cm.

In the embodiment of the apparatus shown schematically in FIG. 2, for example, a 60 Hz, 15,000 V RMS transformer may be employed as the source of the RF power. A Teflon sheet of approximately 0.030 inches in thickness may be used to insulate each electrode. Teflon has a dielectric strength of 430,000 Volts/inch (169,291 V/cm) and a total thickness of approximately 0.060 inches is sufficient to prevent sparks even if a conductive commodity is placed between the electrodes. With an electrode spacing or gap of 4.5 cm and an electric field potential of 15,000 V RMS (i.e. 43,000 V peak-peak), it has been shown that fruit flies (Drosophila melanogaster) can be effectively controlled on grapes and other food commodities within a matter of minutes.

The apparatus 200 can be configured to dimensions large enough to treat multiple pallets or containers 210 of packed table grapes, bush berries, or other food commodity as shown schematically in FIG. 3. In one embodiment, horizontal plates 212a, 212b are separated by approximately 100 cm, which is sufficient distance to treat commercial sized pallets 210. A 60 Hz voltage source 214 of 300 kV RMS, should be sufficient to control fruit flies on grapes or berries, for example.

The voltage source 214 preferably is powered by a conventional power supply 216. In this embodiment, a 4 centimeter thick layer of polyester or polypropylene over the electrodes should adequately protect against destructive arcing effects. Polyester has a dielectric strength of 7 MV/inch or (2.8 MV/cm) and polypropylene has a dielectric strength of 5 MV/inch or (2.0 MV/cm).

Alternatively, solid-state switching supplies may be used instead of the transformer referred to above. The output frequencies of these devices can be from 2 KHz to 60 KHz and the voltages can exceed 160 kV peak-peak. Such solid-state switching supplies can be made smaller and less expensive than transformers. For example, a low cost power source may be provided that can produce frequencies of up to approximately 400 KHz using an array of power MOSFETS. MOSFET devices can easily be wired in parallel in order to achieve higher power handling capabilities. These devices are of interest because they are inexpensive, work over a large range of input voltages, and can handle a wide range of load impedances. By setting up a simple switching network, these devices can provide frequencies up to approximately 400 KHz. MOSFETs can also be wired in parallel to provide any current or power requirement. They can switch power from a DC source, or they can be wired in a bridge to switch AC power if the 60 Hz modulation is a desirable characteristic. The control circuitry is also much simpler than conventional RF sources and may consist of a power output level monitor and control system and a fault detect with auto shutoff. An RF system that is capable of delivering 40 KW at 400 KHz could be built for substantially less than conventional RF power sources.

It can be seen that the size and type of power supply, transformer and electrode dimensions and characteristics can be adapted to provide varying sized treatment spaces that will accommodate existing pallets, storage or field containers.

Accordingly, the low frequency disinfestation method incorporates a source of a low frequency oscillating high voltage electric field to lethally disrupt the metabolism of insect pests that has a frequency that is low enough so as not to add any thermal energy to the host food product.

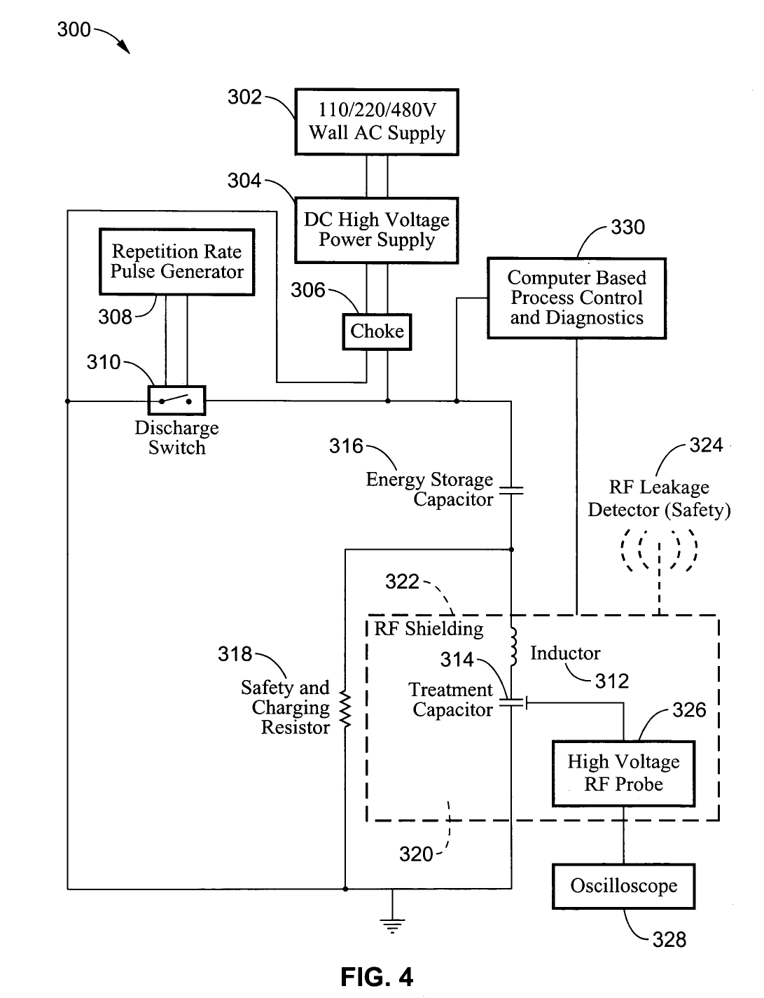

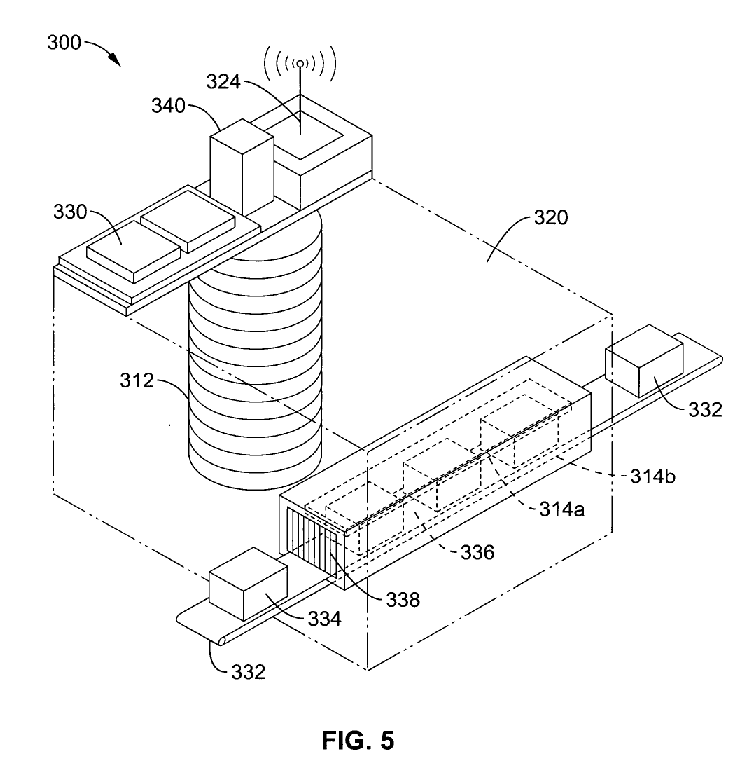

Referring now to FIG. 4 and FIG. 5, an embodiment of a high frequency disinfestation (High-RFD) apparatus 300 is schematically shown. In this embodiment, the apparatus is configured for processing boxes continuously or in small batches. The high frequency process normally operates with frequencies of approximately 1 MHz to approximately 150 MHz and electric fields of preferably greater than approximately 5 kV/cm. The major advantage of the High-RFD process is that the time-of-processing is significantly reduced and therefore appropriate for on-line processing.

Turning now to FIG. 4, the high electric field pulsed RF processing system preferably has a power supply 302 that will operate using municipal power to minimize energy costs. The available municipal power can be either alternating current or direct current. Municipal power is usually available in 110, 220 and 480 Volt sources. A DC high voltage power supply 304 is preferably provided that will produce high voltages of approximately 5 kV or higher. A choke 306 may also be provided in the circuit. The power supply 302 is preferably linked to a repetition rate pulse generator 308 with a discharge switch 310 to permit the modulation of the rate of pulse repetition.

The power supply 304 is connected to an induction coil 312 and opposing plates or electrodes 314. The circuit may also have an energy storage capacitor 316 and a safety and discharge resistor 318 to control voltage fluctuations etc. The coil 312 and plates 314 are preferably housed within a housing 320 that has RF shielding 322 that will not permit the escape of RF waves from the housing 320. In the embodiment shown, the housing 320 also includes a RF leakage detector 324 for safety.

Optionally, the system may have a high voltage probe 326 with an oscilloscope 328 and a computer 330 to monitor the input and output of the system.

In the embodiment shown in FIG. 5, the apparatus 300 has a conveyor 332 that can continuously guide boxes 334 of commodities for processing. The conveyor passes through a chamber 336 that has at least one pair of opposing cavity plates 314a and 314b. The entry and exit ends of the chamber 336 preferably have shielded openings 338 to reduce the escape of RF energy from the chamber during treatment. In one embodiment, the commodity containers 334 are reversibly sealed and may have an anoxic environment. The RF treatment of the contents of the containers 334 may be at frequencies that are lethal or sub-lethal as needed depending on the commodity and the nature of the various pests that are targeted for disinfestation.

Discharging the energy storage capacitor 316 via the discharge switch 310 excites parallel plates 314a and 314b. This energy is then transformed by the inductor 312 and the treatment capacitor 316 into a decaying burst of RF energy. Optionally, the apparatus may have a microprocessor 330, a spectrum analyzer 340, a scope 328 and an RF leakage detector 324 to program and monitor the activity of the apparatus 300. RF pulses are generated in order to release the energy stored in a high-voltage capacitor 316 very rapidly using a high speed, high voltage switch, such as a Thyratron or a spark gap switch.

The distance between the plates 314a and 314b is selected to optimize pest mortality and to minimize injury to the commodity. Variation in the distances is influenced by the type of commodity to be treated in order to reduce the potential for injury to the commodity as well as the type of pests that are present to optimize mortality. For example, in the case of berries treated in conventional polyethylene containers, the gap between plates 314a and 314b and the container 334 would preferably be approximately 4.5 cm. For bulk, shelled almonds, the gap can be approximately 6 cm. For boxes of table grapes the gap may increase to approximately 17 cm.

The inductor 312 and cavity plates 314a and 314b may be connected in series as shown in FIG. 4 or in parallel forming what is commonly known as tank circuit. For example, when an inductor and a capacitor are connected in series and energized by a rapidly changing DC voltage source, oscillations will occur at a characteristic frequency as energy is exchanged between the capacitor and the inductor. Changing the value of the capacitor or the inductor or both in this embodiment may change the frequency of the oscillations.

It will be seen that the various elements of the apparatus can be modulated to provide different processing characteristics for different types of commodities and pests. For example, the geometries of the processing chamber 320, the cavity plates 314 and induction coil 312 can be configured to provide a range of electric field characteristics tailored to a particular setting such as a storage or quarantine setting. Likewise, the power supply and high voltage capacitors may be optimized to provide sufficient field strength and reduce the consumption of electricity.

The High-RFD method preferably uses periodic (non continuous or alternating) pulses of RF energy delivered repeatedly within a parallel-plate chamber 336 using frequencies, pulse shapes, duration and repetition rates that deliver RF power in a manner that causes no detectable thermal energy production in the host commodity. The RF power is preferably pulsed with extremely short duration times (nano to microseconds) and is applied with repetition rates ranging from approximately one to approximately ten thousand pulses per second. Pulses are formed with rapid rise and fall times while pulse duration is maintained as short as possible (i.e. less than milliseconds). Pulses with sinusoidal shapes are preferred. However, pulses with other forms are also adequate. In addition, pulses with a square-wave form are preferred over other types of pulses including, but not limited to, exponential, bipolar, and oscillatory pulses.

The damage to insects from RF treatments appears to be accumulative. By increasing the number of pulses per second, the insects can be controlled in less time of total exposure. For example, a device that can deliver 8 Joules per pulse and a DC power supply rated at 1.2 kW, the maximum pulse repetition rate can be approximately 150 pulses per second. If the DC power supply was rated at 12 kW, the pulse repetition rate could be around 1,500 pulses per second.

Therefore, if the approximate number of pulses needed to control a particular insect on a certain commodity is known, the apparatus can be configured with plate dimensions, energies and pulse frequencies to reduce the total effective time of exposure to a minimum. Alternatively, system optimization can be based upon the most resistant insect or mite contaminant so that the operational and functional features would be applicable to a broader spectrum of infested commodities and pests.

It can be seen that the frequency selection may be very important in the optimal operation of the pulsed RF system. Frequencies, such as 250 kHz, have the distinct advantage of generating high electric fields for a small investment in power per pulse, however these frequencies tend to have serious problems with corona discharge.

Predictably, corona problems are easier to avoid with lower frequencies (<10 kHz), since the RF energy per pulse is also considerably lower than at high frequencies. At lower frequencies, a layer of insulating material such as polypropylene or polyester over the electrodes can effectively limit the current to prevent sparks. Polyester, available as Mylar, has a dielectric strength of 7 MV/in. (2.8 MV/cm), and can be used as a high voltage insulator and spark suppressor in one embodiment.

Frequencies above approximately 6 MHz are also useful, since corona effects and sparks can be managed with thin sheets of Teflon or similar insulating materials. At these higher frequencies, air molecules are not as susceptible to high voltage breakdown because the electric field reverses polarity before an ionizing breakdown path has been established. Under normal circumstances, air has a dielectric strength of 80 kV/in. (31.5 kV/cm).

In another illustration, at 40 MHz, an electric field of greater than approximately 16 kV/cm is preferred to control flies on grapes. In order to achieve that electric field with a gap of approximately 17 cm, a total electric field of approximately 300 kV is desired. One system that meets these conditions is a 150 kV DC Power Supply with a capacitor bank using three 50 kV high voltage capacitors in series, and a high-voltage corona stabilized rotary spark gap.

An alternative method for generating the extremely high voltages needed to treat commodities needing larger gaps (i.e. grapes at approximately 17 cm) would be to lower the frequency and use a Hi-Q inductor to “ring” the voltage up by a factor greater than five. Another advantage with this embodiment is that the pulsed RF system can operate with a DC Power Supply, Storage Capacitor, and Spark Gap working in the 40 to 60 kV range, thus greatly simplifying the engineering, increasing the reliability, and reducing cost of the high voltage components.

The apparatus may also be used for processing thermally tolerant commodities such as dried foods, grains and nursery soils, utilizing continuous, non-pulsed energy within the frequency range allowing controlled thermal effects as well. In this operational mode, using the drifting mechanism in conjunction with “controlled thermal” effects should be balanced to avoid or minimize thermal effects, although the combination of thermal energy (controlled or minimal) with drifting effects may provide synergistic effects against some pests.

The invention may be better understood with reference to the accompanying examples, which are intended for purposes of illustration only and should not be construed in any sense as limiting the scope of the present invention as defined in the claims appended hereto.

To demonstrate the non-thermal disinfestation method with pulsed radio frequencies alone on insects and mites, samples of table grapes, bush berries and almonds were artificially infested with either Thrips (Frankliniella occidentalis); Fruit Flies (Drosophila melanogaster); Ants (Pogonomyrmex subdentata); Aphids (Myzus persicae); Harlequin Bugs (Murgantia histrionics), or Mites (Amblyseius cucumeris and Tetranychus urticae). Some test subjects included different stages of the life cycle from egg to larva, pupa, juvenile and adult. Mortality was assayed both at the end of exposure as well as twenty-four hours after exposure to the treatments and compared with control subjects. Visual and microscopic observations of anatomical changes and lack of motion confirmed mortality. For eggs and larva, mortality effects were observed over several days due to longer biological time cycles. No temperature changes were observed in the host commodities for either the control or treated subjects.

### EXAMPLE 1

The cumulative results of the low frequency RF disinfestation treatments at various field strengths and frequencies are shown in Table 1. Low frequency RF was arbitrarily defined as a using frequencies ranging from approximately 10 Hz to 1 MHz with a preferred field intensity of greater than approximately 5 kV/cm.

It can be seen that fruit flies and ants are particularly vulnerable to treatments with a field strength of 12 kV/cm and frequency of 60 Hz. Mortality of Thrip adults and pupas was highest when the frequency was within the range of approximately 300 to 350 kHz with a field intensity of between 23 kV/cm and 35 kV/cm.

### EXAMPLE 2

Table 2 provides the cumulative results of the high frequency RF treatments for fruit flies on table grapes, almonds, blackberries and blueberries at a range of field strengths and radio frequencies. It can be seen that the frequency and field strength can be manipulated to identify the optimum fields and frequencies for a particular pest. Likewise, field strengths and frequencies can be selected to control multiple pests in single host commodity.

It can also be seen that both Low-RFD and High-RFD processes for fresh fruits and vegetables, works faster with oscillating field frequencies of less than approximately 150 MHz. For large volume treatments (batches) with Low-RFD, the process works better with frequencies less than approximately 0.1 MHz. For the on-line treatment with the High-RFD process, it reaches highest efficiency within the frequency range of approximately 5 MHz to 60 MHz.

### EXAMPLE 3

In the treatment of liquids such as fruit juices, RF power can be applied directly to the commodity through inert conductive plates made of material such as Stainless Steel. Even though a commodity such as apple juice has a conductivity of 2.19 mS/cm, the major portion of the heat generated is due to dielectric effects at RF frequencies, rather than resistive effects. There is also no observed electrolysis of the liquid at RF frequencies. Measurements made of a test cell consisting of two Stainless Steel plates 5 cm×3.5 cm separated by 2 cm of apple juice show a capacitance of 80 ufd and an ESR (equivalent series resistance) of 75 Ω. The measurements were made with a Sencore LC103 ReZolver, 3200 Sencore Drive, Sioux Falls, SD. Based on the conductivity, the cell has a DC resistance of 570 Ω. Even with stainless steel plates in direct contact with the apple juice, The AC impedance is still much lower than the DC resistance. This effect lends itself well to RF treatments including heating. The AC impedances are also low enough that they can be matched well using conventionally available voltages, thus reducing the equipment costs.

An alternative to the above-described method for treating fluids with RF is to place a thin insulator over one of the electrodes. The two plates, the insulator, and the fluid form a component closely resembling an ideal capacitor. By carefully selecting the dimensions of this capacitor and connecting it to an appropriate inductor, this system may be made to resonate at the switching frequency of the RF generator, maximizing the dielectric losses of the fluid.

The described system could also be easily run in pulsed mode as well as continuous switching mode. The advantage with pulsed mode would be for treating commodities with low internal conductivity, such as fishmeal. In this fashion, the commodity would serve as the dielectric media between two plates forming a capacitor. By connecting a suitable inductor to the plates, a resonant cavity is formed. The inductor and the size and spacing of the plates can be selected so as to cause the cavity to resonate at any desired frequency according to the formula:

$$Fr = \frac{1}{2 π \sqrt{LC}}$$

Here, Fᵣ is the resonant frequency, L is the inductance of the coil in Henrys, and C is the capacitance formed the plates and the commodity in farads.

The RF generator need only by pulsed at a repetition rate necessary to offset the steady decay of the ringing of the resonant circuit. The rate of decay will be determined by the dielectric loss of the commodity and any other resistive or reactive losses of the resonant circuit. The pulsed excitation could be introduced at a tap of the coil, thereby allowing for significant voltage multiplication of the initial pulse.

Accordingly, an apparatus and method are provided for disinfestation of temperature sensitive and other commodities using short duration, high peak power radio frequency pulses and intense electric fields to preferentially induce drift of conduction charges, spot heating and other mortal damage to infesting insects and mites without raising the temperature of the host commodity. The method is non-contact, residue free and effective with all biological stages of an infesting insect, mite or other biological pest including egg, pupa, larvae, juvenile and adult forms. The apparatus and methods of the invention are an effective alternative to methyl bromide fumigation that does not leave any toxic residues or damage the cosmetic appearance or flavor of the commodity.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE 1
Summary of Representative Results with Low Frequency Pulsed RF
Disinfestation Method

| Pulsed RF System | | | Thermal | | Mortality | |
|---|---|---|---|---|---|---|
| | E Field | | Effects | Insect/Mites | | Instant + Delayed |
| F | KV/cm | Host Product | (ΔT° C.) | (Species/Stage) | Instant | at <24 h |
| 60 Hz | 12 | Table | None | Drosophila melanogaster | 100% | 100% |
| | | Grapes | | (Fruit Flies) | | (At 15 min) |
| 60 Hz | 12 | Blueberries | None | Drosophila melanogaster | 100% | 100% |
| | | | | (Fruit Flies) | | (At 15 min) |
| 60 Hz | 12-15 | Table | None | Ants (Pogonomyrmex | 100% | 100% |
| | | Grapes | | subdentata) | | (At 20 min) |
| 80 kHz | 22 | Blueberries | None | Thrips (pupae) | >50% | <80% |
| 200 kHz | 35 | Table | None | Platynota stultana | Not | >99% |
| | | Grapes | | (Leaf Rollers) | Observed | |
| | | | | (Eggs) | | |
| 220 kHz | 36 | Blueberries | None | Thrips (Pupae) | >85% | >99% |
| 240 kHz | 26 | Shelled | None | Platynota stultana | Not | >90% |
| | | Almonds | | (Leaf rollers) | Observed | |
| | | | | (Eggs) | | |
| 240 kHz | 26 | Shelled | None | Lepidoptera (Eggs) | Not | >99% |
| | | Almonds | | | Observed | |
| 350 kHz | 16.5 | Table | None | Drosophila melanogaster | >80% | >95% |
| | | Grapes | | (Fruit Flies) | | |
| 350 kHz | 23 | Blueberries | None | Frankliniella occidentalis | >90% | >99% |
| | | | | (Thrips) | | |
| 500 kHz | 15-20 | Blueberries | None | Drosophila melanogaster | 80-90% | 100% |
| | | | | (Fruit Flies) | | |
| 500 kHz | 15-20 | Table | None | Drosophila melanogaster | 80-90% | 100% |
| | | Grapes | | (Fruits Flies) | | |

TABLE 2
Summary of Representative Results with High-Frequency Pulsed RF
Disinfestation Method

| Pulsed RF System | | | Thermal | | Mortality | |
|---|---|---|---|---|---|---|
| F | E Field | | Effects | Insect/Mites | | Instant + Delayed |
| (MHz) | KV/cm | Host Product | (ΔT° C.) | (Species/Stage) | Instant | at <24 h |
| 6 | 20 | Table | None | Drosophila melanogaster | 100% | 100% |
| | | Grapes | | (Fruit Flies) | | |
| 6 | 20 | Almonds | None | Drosophila melanogaster | 100% | 100% |
| | | | | (Fruit Flies) | | |
| 6.5 | 65 | Table | None | Drosophila melanogaster | 100% | 100% |
| | | Grapes | | (Fruit Flies) | | |
| 6.5 | 38 | Table | None | Drosophila melanogaster | 100% | 100% |
| | | Grapes | | (Fruit Flies) | | |
| 6.5 | 30 | Table | None | Drosophila melanogaster | >60% | 100% |
| | | Grapes | | (Fruit Flies) | | |
| 28 | 19 | Blueberries | None | Drosophila melanogaster | >80% | >95% |
| | | | | (Fruit Flies) | | |
| 38 | 17 | Table | None | Drosophila melanogaster | >90% | >95% |
| | | Grapes | | (Fruit Flies) | | |
| 38 | 17 | Blueberries | None | Drosophila melanogaster | >90% | >95% |
| | | | | (Fruit Flies) | | |
| 40 | 20 | Table | None | Drosophila melanogaster | >90% | >95% |
| | | Grapes | | (Fruit Flies) | | |
| 40 | 20 | Almonds | None | Drosophila melanogaster | >90% | >95% |
| | | | | (Fruit Flies) | | |

What is claimed is:

1. A method for disinfestation of a commodity, comprising:
exposing an infested commodity to a non-thermal, pulsed, high intensity electric field radio frequency for less than approximately 24 hours.

2. A method as recited in claim 1, wherein said radio frequency comprises:
a low frequency;
said low frequency ranging from approximately 10 Hz to approximately 1 MHz.

3. A method as recited in claim 1, wherein said radio frequency comprises:
a high frequency;
said high frequency ranging from greater than approximately 1 MHz to approximately 150 MHz.

4. A method as recited in claim 1, wherein said pulsed radio frequency has a repetition rate ranging from approximately 1 Hz to approximately 10 KHz.

5. A method as recited in claim 1, wherein said pulses have short duration times of less than one second.

6. A method as recited in claim 1, wherein said pulses have rapid rise times and short decay times whereby RF peak power can be maximized.

7. A method as recited in claim 1, wherein said pulses comprise square shaped pulses.

8. A method as recited in claim 1, wherein said pulses comprise sinusoidal shaped pulses.

9. A method as recited in claim 1, wherein said high intensity electric field comprises:
an electric field of greater than approximately 5 kilovolts per centimeter.

10. A method as recited in claim 1, wherein said high intensity electric field comprises an electric field of approximately 12 kilovolts per centimeter when said radio frequency is approximately 60 Hz.

11. A method as recited in claim 1, wherein said high intensity electric field comprises an electric field ranging from approximately 20 kilovolts per centimeter to approximately 70 kilovolts per centimeter when said radio frequency is approximately 6 MHz.

12. A method as recited in claim 1, further comprising:
optimizing a duty cycle, electric field strength and frequency to the sensitivity of a pest to exposure to radio frequency energy.

13. A method as recited in claim 1, further comprising:
optimizing a duty cycle, electric field strength and frequency to the sensitivity of a pest and a host commodity to exposure to radio frequency energy.

14. A method for disinfesting biological pests from a commodity, comprising:
exposing an infested commodity to a first high intensity radio frequency for a first duration; and
exposing said commodity to a second high intensity radio frequency for a second duration;
wherein the temperature of said commodity does not substantially change during exposure.

15. A method as recited in claim 14, wherein said radio frequencies are pulsed.

16. A method as recited in claim 15, wherein said pulsed radio frequencies have a repetition rate ranging from approximately 1 Hz to approximately 10 KHz.

17. A method as recited in claim 14, wherein said pulses have short duration times of less than approximately one second.

18. A method as recited in claim 14, wherein said pulses have rapid rise times and short decay times whereby RF peak power can be maximized.

19. A method as recited in claim 14, wherein said pulses comprise square shaped pulses.

20. A method as recited in claim 14, wherein said pulses comprise sinusoidal shaped pulses.

21. A method as recited in claim 15, wherein said first and second radio frequencies comprise:
low frequencies, said low frequencies ranging from approximately 10 Hz to approximately 1 MHz.

22. A method as recited in claim 15, wherein said first and second radio frequencies comprise:
high frequencies;
said high frequencies ranging from greater than approximately 1 MHz to approximately 150 MHz.

23. A method as recited in claim 15:
wherein said first radio frequency comprises a low frequency, said low frequency ranging from approximately 10 Hz to approximately 1 MHz; and
wherein said second radio frequency comprises a high frequency, said high frequency ranging from greater than approximately 1 MHz to approximately 150 MHz.

24. A method as recited in claim 15:
wherein said first radio frequency comprises a high frequency, said high frequency ranging from approximately 1 MHz to approximately 150 MHz; and
wherein said second radio frequency comprises a low frequency, said low frequency ranging from approximately 10 Hz to less than approximately 1 MHz.

25. A method as recited in claim 14, wherein said high intensity radio frequencies have an electric field comprising:
an electric field of greater than approximately 5 kilovolts per centimeter.

26. A method as recited in claim 14, wherein said first high intensity radio frequency has an electric field of approximately 12 kilovolts per centimeter when said first radio frequency is approximately 60 Hz.

27. A method as recited in claim 14, wherein said first high intensity radio frequency has an electric field ranging from approximately 20 kilovolts per centimeter to approximately 70 kilovolts per centimeter when said first radio frequency is approximately 6 MHz.

28. A method as recited in claim 14, further comprising:
optimizing a duty cycle, electric field strength and frequency of said first radio frequency exposure to the sensitivity of an adult form of a pest to exposure to radio frequency energy.

29. A method as recited in claim 14, further comprising:
optimizing a duty cycle, electric field strength and frequency of said first radio frequency exposure to sensitivity of a larval form of a pest to exposure to radio frequency energy.

30. A method as recited in claim 14, further comprising:
optimizing a duty cycle, electric field strength and frequency of said first radio frequency exposure to sensitivity of an egg form of a pest to exposure to radio frequency energy.

31. A method as recited in claim 14, further comprising:
optimizing a duty cycle, electric field strength and frequency of said first and second frequency exposures to sensitivity of a pest and a host commodity to exposure to radio frequency energy.

32. A method as recited in claim 14, wherein said first radio frequency is optimized to disinfest egg forms of a pest and said second radio frequency is optimized to disinfest adult forms of a pest.

33. A method as recited in claim 14:
wherein said first radio frequency is configured to produce ozone; and
wherein said second radio frequency is optimized to disinfest adult forms of a pest.

34. A method as recited in claim 14, wherein said first duration and said second duration combined do not exceed approximately 24 hours.

35. A method for residue free disinfestation of an article, comprising:
treating an article with pulsed high intensity radio frequency electromagnetic radiation at a plurality of different frequencies; and
controlling the temperature of said article over a course of treatment.

36. A method as recited in claim 35, wherein said temperature of said article is controlled by modulating the radio frequency, pulse rate or electric field intensity during treatment.

37. A method as recited in claim 35, wherein said article is treated with high intensity radio frequency electromagnetic radiation that has an electric field intensity of greater than approximately 5 kilovolts per centimeter.

38. A method as recited in claim 35, wherein said pulses have rapid rise times and short decay times whereby RF peak power can be maximized.

39. A method as recited in claim 35, wherein said pulses comprise square shaped pulses.

40. A method as recited in claim 35, wherein said pulses comprise sinusoidal shaped pulses.

41. A method as recited in claim 35, wherein said pulsed radio frequency electromagnetic radiation has a pulse rate of more than approximately one pulse per second.

42. A method as recited in claim 35, wherein said pulsed radio frequency electromagnetic radiation has a pulse rate of between approximately 10 pulses per second and approximately 1 million pulses per second.

43. A method as recited in claim 35, wherein said pulsed radio frequency electromagnetic radiation has a pulse rate of between approximately 1 million pulses per second and approximately 10 million pulses per second.

44. A method as recited in claim 35, wherein said electromagnetic radiation comprises radio waves with a frequency ranging between approximately 10 Hertz and approximately 1 Megahertz.

45. A method as recited in claim 35, wherein said electromagnetic radiation comprises radio waves with a frequency ranging between approximately 1 Megahertz and approximately 150 Megahertz.

46. A method as recited in claim 35, wherein said radio frequency comprises:
a radio frequency and an electric field strength configured to produce ozone and oxygen radicals from ambient air.

47. A method as recited in claim 35, wherein said exposure to electromagnetic radiation at a plurality of radio frequencies comprises:
exposure to a first radio frequency for a first duration;
exposure to a second radio frequency for a second duration; and
exposure to a third radio frequency for a third duration.

48. A method as recited in claim 35, wherein said first, second and third radio frequencies comprise:
a low frequency, said low frequency ranging from approximately 10 Hz to approximately 1 MHz.

49. A method as recited in claim 35, wherein said first, second and third radio frequencies comprise:
a high frequency, said high frequency ranging from approximately 1 MHz to approximately 150 MHz.

50. A method as recited in claim 35, wherein said first radio frequency comprises a low frequency, said low frequency ranging from approximately 10 Hz to approximately 1 MHz; and said second radio frequency comprises a high frequency, said high frequency ranging from approximately 1 MHz to approximately 150 MHz.
